(19) United States
(12) Patent Application Publication
(10) Pub. No.: US 2020/0185069 A1
(43) Pub. Date: Jun. 11, 2020

(54) MEDICAL CODING QUALITY CONTROL

(71) Applicant: 3M INNOVATIVE PROPERTIES COMPANY, St. Paul, MN (US)

(72) Inventors: Gary M. Jeppson, Sandy, UT (US); Scott F. Cass, Cumberland Foreside, ME (US); David T. Dundon, Chantilly, VA (US); Myung H. Kim, Woodbridge, VA (US)

(21) Appl. No.: 16/619,376

(22) PCT Filed: Jun. 1, 2018
(86) PCT No.: PCT/IB2018/053957

§ 371 Date: Dec. 4, 2019

Related U.S. Application Data
(60) Provisional application No. 62/516,319, filed on Jun. 7, 2017.

(51) Int. Cl. G16H 10/60; G16H 15/00; G16H 70/20

(52) U.S. Cl. CPC G16H 10/60; G16H 70/20; G16H 15/00

(57) ABSTRACT

A system and method for auditing medical records to determine the coding accuracy of medical coding professionals. The system assigns an expected coding accuracy level to each medical coding professional, establishes a confidence level and a margin of error for each expected coding accuracy level, determines code populations for each medical coding professional, determines a sample size for each medical coding professional for each code population, randomly retrieves medical records to obtain, for audit, samples of codes assigned by the respective medical coding professionals, determines if the codes obtained from the retrieved medical records were assigned correctly, and calculates an accuracy for each medical coding professional based on the number of correct code assignments by each respective medical coding professional.

### TECHNICAL FIELD

The invention relates to medical coding quality control systems and techniques.

### BACKGROUND

When a healthcare provider interacts with a patient in a hospital setting, the provider memorializes the encounter, usually by typing or dictation. The provider may, for instance, memorialize the condition of the patient, the treatment plan, and what was done to the patient for treatment. The resultant encounter-related documentation may subsequently be reviewed by documentation review specialists, who may read through, update and correct the encounter-related documentation. Encounter-related documentation may be reviewed by billing specialists to determine the most effective combination of billing codes for each encounter.

### SUMMARY

In general, this disclosure describes a system and method for conducting a quality review of medical records to determine coding accuracy of medical coding professionals in a healthcare organization.

In some example approaches, a method of auditing medical records to determine the coding accuracy of medical coding professionals comprises assigning an expected coding accuracy level to each of a plurality of medical coding professionals; establishing a confidence level and a margin of error around each expected coding accuracy level; determining a code population for each medical coding professional and for each of one or more code types, each respective code population including codes that are subject to audit, that are of the same code type and that were assigned by the respective medical coding professional, the code population having a size that represents the number of codes therein; determining a sample size SS(i) for each medical coding professional i for each code population, the sample size based on the expected coding accuracy level, the confidence level and the margin of error associated with the respective medical coding professional; randomly retrieving medical records to obtain, for audit, approximately SS(i) samples of codes assigned by each respective medical coding professional i; determining if the codes obtained from the retrieved medical records were assigned correctly; and calculating an accuracy for each medical coding professional based on the number of correct code assignments by each respective medical coding professional.

In another example approach, an auditing system includes a memory, a network interface, and at least one processor connected to the memory and the network interface, wherein the memory includes instructions that, when executed by the at least one processor, cause the processor to audit medical records to determine the coding accuracy of medical coding professionals, wherein the auditing includes assigning an expected coding accuracy level to each of a plurality of medical coding professionals; establishing a confidence level and a margin of error around each expected coding accuracy level; determining a code population for each medical coding professional and for each of one or more code types, each respective code population including codes that are subject to audit, that are of the same code type and that were assigned by the respective medical coding professional, the code population having a size that represents the number of codes therein; determining a sample size SS(i) for each medical coding professional i for each code population, the sample size based on the expected coding accuracy level, the confidence level and the margin of error associated with the respective medical coding professional; randomly retrieving medical records to obtain, for audit, approximately SS(i) samples of codes assigned by each respective medical coding professional i; determining if the codes obtained from the retrieved medical records were assigned correctly; and calculating an accuracy for each medical coding professional based on the number of correct code assignments by each respective medical coding professional.

The details of one or more aspects of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an example electronic health record capture and coding system, according to one aspect of the disclosure.

FIG. 2 is a flowchart illustrating an example method of auditing the performance of a medical coding expert, according to one aspect of the disclosure.

FIGS. 3A-3C illustrate a method of determining an appropriate sample size to audit a medical coding professional, according to one aspect of the disclosure.

FIG. 4 is a flowchart illustrating another example method of auditing the performance of a medical coding expert, according to one aspect of the disclosure.

FIG. 5 illustrates an attribute assessment agreement, according to one aspect of the disclosure.

### DETAILED DESCRIPTION

When a healthcare provider sees a patient in either an outpatient clinic or during an office visit (e.g., a patient encounter), the provider typically performs an evaluation of the patient, the patient's medical history and/or the patient's current medical condition. The provider may also perform a medical procedure on the patient during the patient encounter or prescribe treatment related to the patient's medical condition. The healthcare provider typically documents the patient encounter during or soon after the encounter, writing or dictating notes regarding the patient's condition, treatments, etc. The encounter-related documentation may be used to update an electronic health record (EHR) associated with the patient. The electronic health record is also known as an electronic medical record. The encounter-related documentation may also serve as the basis for a claim for reimbursement for the services performed during the patient encounter.

To be reimbursed for medical services, a healthcare provider may submit a claim for the services performed during the patient encounter. Typically, such a claim is accompanied by codes generated based on the patient encounter. Often, healthcare organizations employ experts who review encounter-related documentation and determine the most effective combination of billing codes for each encounter. Coding is the medical business practice of matching the clinical documentation in a patient's record to numerical and alphanumerical codes for reimbursement of services. The coding process is usually done by medical coding experts reviewing the encounter-related documentation, by processors using natural language processing (NLP) algorithms to review the encounter-related documentation, or by a combination of the two.

For example, the patient evaluation and the medical decisions made by the physician during the patient encounter may be submitted for billing as an E/M code. An E/M level code may include details on certain components that are combined to provide the E/M level code. Example components of the code may include a history, a physical examination and medical decision making.

Procedures that are performed during the patient encounter may be submitted for billing as CPT codes. The physician may also submit appropriate diagnosis codes (e.g., ICD-10 codes) related to the patient's condition, which may accompany the E/M and CPT codes. One or more of these generated codes for the patient encounter may be submitted to the clinic or office billing system of the healthcare provider for submission to the appropriate insurance payer.

Typically, the E/M code has several levels, depending on how in-depth, time-consuming and involved the physician evaluation of the patient was for that patient encounter. The criteria involved in selecting the appropriate level for each visit are complex and broken down into multiple components and sub-components related to what the physician did during the patient encounter. Physicians and their clinic or office staff routinely face issues of physicians either under-coding (i.e. selecting a lower E/M level than is appropriate for the level of services rendered) or over-coding (i.e. selecting an E/M level above what is appropriate for the level of services rendered or documented). Under-coding may result in lower compensation for the physician and clinic; over-coding may result in additional administrative burden, payer enforced penalties, and other sanctions. Healthcare organizations often, therefore, employ medical coding experts to review the codes selected by the physician against the underlying documentation. A medical coding expert may be able to use such a review to correct errors in documenting and coding the patient encounter and in the billing information submitted for reimbursement.

Many healthcare organizations have an EHR system, which maintains an EHR for each patient served by the healthcare organization, typically in a digital format. Each EHR contains the medical record for a patient; the information contained in the EHR for each patient is, however, usually spread across multiple documents and reports, and may lack a cohesive, validated and updated summary of the patient and his or her conditions. A physician spends a significant amount of time reviewing EHRs, determining treatment plans, issuing orders and documenting the encounters with their patients.

EHR systems can simplify the process of capturing billing and diagnostic codes. Physicians may use an EHR system in their clinic or office to generate E/M codes, CPT codes, and/or diagnosis codes from a series of pick-lists, check-boxes and drop-down menu items, which the EHR system uses to automatically calculate the E/M level. Physicians typically add a clinical documentation note (e.g., clinical documentation) for the patient encounter to further detail the services that were provided by the physician and/or clinic. A healthcare organization with an EHR system may employ medical coding experts to review the codes selected by the physician, and the calculated E/M level, against the underlying documentation. A medical coding expert may be able to use such a review to correct errors in documenting and coding in the patient's EHR and in the billing information submitted for reimbursement.

As noted above, the quality of the coding is crucial. Under-coding may result in missed reimbursement opportunities, while over-coding may result in charges and fines for the healthcare organization. In fact, a single instance of incorrect coding may end up costing a medical provider thousands of dollars in Medicare reimbursements. In many healthcare organizations, the medical coding experts serve as a last line of defense against under-coding and over-coding in reimbursement claims. It is critical, therefore, to ensure the quality of their medical coding. The systems and methods disclosed herein show examples of systems designed to facilitate efficient quality review of the coding of encounter-related documentation.

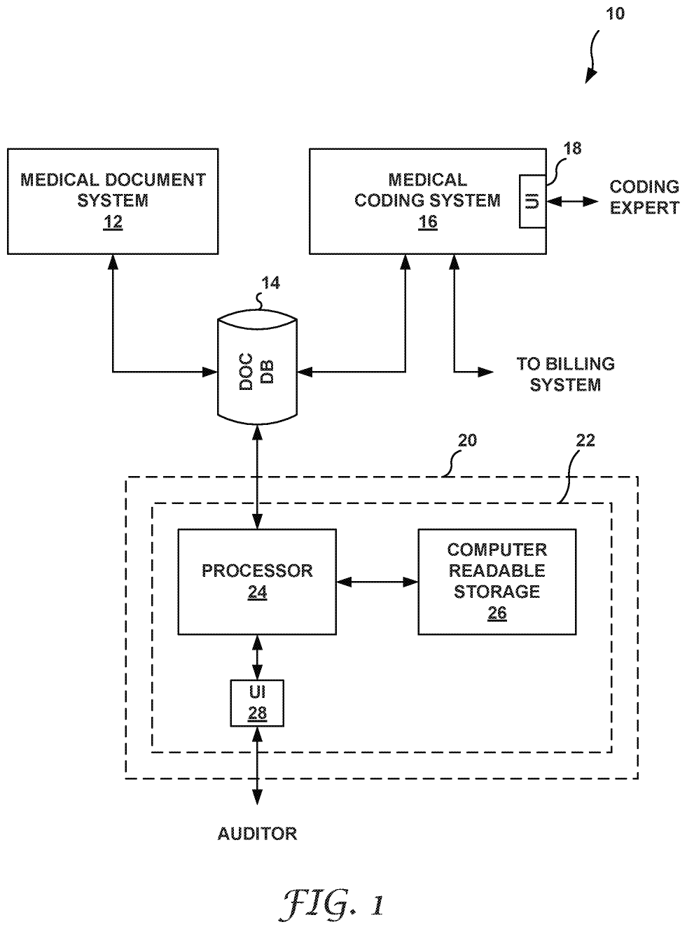

FIG. 1 is a block diagram illustrating an example electronic health record capture and coding system, according to one aspect of the disclosure. In the example of FIG. 1, electronic health record and coding system 10 includes a medical document capture system 12 connected to a medical document database 14 and a medical coding system 16 connected to medical document database 14. In some example approaches, medical document capture system 12, medical coding system 16 and medical document database 14 form an EHR system as detailed above. In some example approaches, healthcare organization coding system 16 includes a user interface (UI) 18 used by a medical coding expert to assign the appropriate codes to each medical document. In some example approaches, the documents stored in medical document database 14 include electronic health records, encounter-related documentation and documents such as problem lists and billing records that are derived from encounter-related documentation.

A physician or other healthcare provider creates documents (such as clinical notes) for a patient during a patient visit; medical document capture system 12 then stores the information from the notes to medical document database 14. The doctor may create the documents, for instance, through dictation-transcription, or the physician may, for instance, enter the information directly into the medical document database 14 via medical document capture system 12. This process may result in new documentation or clinical notes that become part of the permanent medical record for that patient in the EHR system. In some example approaches, the captured information, along with information about the patient contained in other hospital systems such as laboratory data, test results or medications as well as patient admission, discharge and transfer (ADT) information is processed via a natural language processor to extract information related to diagnosis and treatment of the patient. Any portion of this information received by the NLP may be considered medical documentation associated with the patient.

In some example approaches, medical document capture system 12 includes an NLP-driven automated analysis process. In one such example approach, the NLP-driven automated analysis process assembles all available information about a patient's case into a multi-document view of the patient called a “case model,” which may be described as a broad summary about that patient's case or history, and includes patient encounter-related information. This analysis may include identifying and tagging, within every document or data source, each diagnosis, symptom, vital sign, or other patient information, as well as each test, lab, or procedure performed. This analysis may also include determining whether each element in the case model is current for the visit or encounter, or whether each element is historical (i.e., from a past encounter), or is related to a familial history or linkage. In one such example approach, each relevant piece of information about the patient's current, historic, or familial medical history (e.g., documented items) may then be mapped by the NLP automated analysis process to a concept code called a concept identification code. The concept identification code is an intermediary code set that is mapped to and from other commonly used code sets. Each concept code may define or represent a medical concept. The common identifier codes for each patient, along with the relationships between each common identifier codes, are then stored in the case model for that patient in medical document database 14.

In some example approaches, the common identification codes are part of what is termed a healthcare data dictionary (HDD). Each of the concept identification codes may then be then mapped, or linked, to other available industry coding sets or terminology standards, such as the International Classification of Diseases (e.g., ICD-10 codes) or the SNOMED-CT codes. Mapping every piece of information in a patient's medical record to a concept identification code, allows for ready translation of any one code or term from one standard, to any other code or term from another standard. In one such example approach healthcare organization coding system 16 reviews the codes assigned by the NLP-driven automated analysis process, generates corrected codes, annotates the medical records to reflect the corrected codes and stores the annotated documents in medical document database 14.

In some example approaches, the NLP-driven automated analysis process performs natural language processing of each document looking for variations of key words and phrases, as well as information specific to, for instance, one or more ICD codes, annotating the medical document before storing the document to database 14 for subsequent processing by healthcare organization coding system 16. For example, if a given term is found, that term may be suggestive of a corresponding ICD code. The information is therefore associated with the term. In one such example approach, the association is documented in a new annotated version of the document in a markup language that allows for the embedding of metadata with terms, such as HTML, or a variant of XML. Natural language processing in general and its application to the computer-assisted coding of medical record data are described by Wolniewicz in Computer-assisted Coding and Natural Language Processing, https://multimedia.3 m.com/mws/media/7568790/3m-cac-and-nlp-white-paper.pdf, the description of which is incorporated herein by reference. Wolniewicz discusses the use of tokenization, sentence and structure detection, part-of-speech (POS) tagging, normalization, named entity resolution, parsing, negation and ambiguity detection and semantics in natural language processing of medical documents. Wolniewicz also describes the use of the Unstructured Information Management Architecture (UIMA) as an appropriate technical platform used to supply these capabilities.

In some example approaches, the NLP-driven automated analysis process implements statistical natural language processing. Statistical NLP means that one or more processors 18 learn the mappings for the NLP components as statistical relationships by processing many examples. The accuracy of a statistical model increases with the volume of data available for learning. In fact, the performance of a deployed system 10 will improve after deployment as the system learns the codes most often selected. Statistical methods, however, required a very large annotated data set to use for training. In one such example approach, machine learning NLP is implemented on the open-source UIMA software platform, a standardized and integrated NLP solution.

In one such example approach, processor 18 is configured as a machine learning processor to parse medical documents into tokens and then analyze the tokens to generate skip-grams. A skip-gram is a way of modeling language. A skip-gram is based on a construct referred to as an n-gram. An n-gram is a consecutive subsequence of length n of some sequence of tokens w₁ . . . wₙ. A k-skip-n-gram is a length-n subsequence having components that occur at distance at most k from each other. As an example, for the phrase “the quick brown fox jumps over the lazy dog,” the set of all 1-skip-2 grams comprises: “the brown,” “quick fox,” “brown jumps,” “fox over,” “jumps the,” “over lazy,” and the dog,” as well as all the 2-grams (also referred to as bigrams), e.g., “the quick,” “quick brown,” etc. Skip-grams may be more useful relative to n-grams for analyzing word data due to the data sparsity associated with n-grams.

In some example approaches, processor 18 implements an algorithm that examines “skip-grams” of tokens from medical documents and builds a “trie” data structure (also referred to as a prefix tree) via the skip-grams. Processor 18 may determine, based on the nodes of the trie, rules for associating medical codes with terms and phrases in medical documents. Negative sampling models and models that treat documents as bags of words may be used as well.

In another example approach, medical coding system 16 includes the NLP-driven automated analysis process. In one such example approach, the NLP-driven automated analysis process analyzes the case model as detailed above, identifying and tagging, within every document or data source, each diagnosis, symptom, vital sign, or other patient information, as well as each test, lab, or procedure performed. This analysis may also include determining whether each element in the case model is current for the visit or encounter, or whether each element is historical (i.e., from a past encounter), or is related to a familial history or linkage. In one such example approach, each relevant piece of information about the patient's current, historic, or familial medical history (e.g., documented items) may then be mapped by the NLP automated analysis process to a concept code called a concept identification code. The concept identification code is an intermediary code set that is mapped to and from other commonly used code sets. Each concept code may define or represent a medical concept. The common identifier codes for each patient, along with the relationships between each common identifier codes, are stored in the case model for that patient in medical document database 14.

One or more analyses may be performed on the information in the case model which, in some cases, may result in multiple outputs, such as Computer-Assisted Physician Documentation (CAPD) queries, problem list suggestions (e.g., potential medical problems), specialist queries (i.e., Clinical Document Improvement (CDI) queries), as well as high-risk patient alerts, ICD-9 codes, ICD-10 codes, or other types of information related to the patient. Medical document capture systems, medical document databases, NLP-driven automated analysis processes and coding processes are described in further detail in U.S. patent application Ser. No. 14/771,852, entitled SYSTEMS AND METHODS FOR REQUESTING MEDICAL INFORMATION, filed Feb. 28, 2014, and in U.S. patent application Ser. No. 15/120,140, entitled COMPUTER-ASSISTED MEDICAL INFORMATION ANALYSIS, filed Feb. 18, 2015, the discussions of which are incorporated herein by reference.

Medical document database 14 may include one or more memories, repositories, databases, hard disks or other permanent storage, or any other data storage devices. Medical document database 14 may be included in, or described as, cloud storage. In other words, information stored on medical document database 14 and/or instructions that embody the techniques described herein may be stored in one or more locations in the cloud. A medical document capture system 12 or a medical coding system 16 may access the cloud and retrieve or transmit data as requested by an authorized user via a user interface such as user interface 18. In some examples, medical document database 14 may include Relational Database Management System (RDBMS) software. In one example, medical document database 14 may be a relational database and accessed using a Structured Query Language (SQL) interface. Medical document database 14 may alternatively be stored on a separate networked computing device and accessed by medical document capture system 12 or by medical coding system 16 through a network interface or system bus. Medical document database 14 may in other examples be an Object Database Management System (ODBMS), Online Analytical Processing (OLAP) database or other suitable data management system.

Returning to FIG. 1, In some example approaches, electronic health record capture and coding system 10 further includes a coding quality auditing system 20. In the example approach of FIG. 1, coding quality auditing system 20 is connected to medical document database 14 and retrieves from database 14 samples of coding performed by medical coding experts within the healthcare organization. In some example approaches, the samples are reviewed by auditors via user interface 28 to verify that the coding accuracy goals of the organization are being met.

In some example approaches, coding quality auditing system 20 includes a computer 22 having one or more processors 24 connected to computer readable storage 26 and to user interface 28. In one such example approach, instructions stored in computer readable storage 26, when executed by the one or more processors 24, cause the processors to review the coding efforts of a medical coding expert for accuracy, as will be discussed in further detail below. Computer readable storage 26 may include one or more non-volatile memories, hard disks or other permanent storage, or any other data storage devices. Computer readable storage 26 may include cloud storage.

In some example approaches, coding quality auditing system 20 establishes a connection across a network to document database 14. The network may be a local network, a wide area network, or the Internet. In one such example approach, coding quality auditing system 20 reads the medical records from document database 14.

In some example approaches, coding quality auditing system 20 establishes a connection across a network to medical coding system 16, and through medical coding system 16, to document database 14. The network may be a local network, a wide area network, or the Internet. In one such example approach, coding quality auditing system 20 reads the medical records from document database 14.

In some example approaches, coding quality auditing system 20 establishes a connection across a network to a medical document system 12 having a medical coding system 16 and a document database 14. The network may be a local network, a wide area network, or the Internet. In one such example approach, coding quality auditing system 20 reads the medical records from document database 14 either directly, or through one of medical document system 12 or a medical coding system 16.

Most healthcare organizations perform some level of quality testing of medical coding results. Many such organizations, however, perform inefficient and, sometimes, unnecessary tests when checking the quality of medical coding. Common industry medical coding quality control problems include inaccurate sampling, lack of fidelity of quality scores, unnecessary post-audit reviews and little to no standardization of the audit process.

With respect to the inaccurate sampling problem, many organizations rely on “guessing” to determine the appropriate coder sample size to audit. Such an approach leads to high costs and uncertain quality results; organizations tend to oversample as the normal intuition is to increase the audit sample as the size of the population of codes increases. This is faulty logic.

With respect to the lack of fidelity in quality scores, there often is uncertainty regarding the performance of individual medical coding experts (or the quality of NLP coding programs) or the meaning of a quality score. For instance, without statistical science, how does one know if the statistical sample was adequate to satisfactorily infer the conclusions reached on medical coding expert performance?

With respect to unnecessary post-audit reviews, many organizations waste resources performing unnecessary rebuttals on audit results that fall below a client established standard but that are still acceptable (i.e., that are still within a statistical margin of error (MOE)).

Finally, there is little to no standardization of the audit process. That is, typically, the healthcare organization fails to ensure that auditing standards are “calibrated” among the different medical coding auditors and are applied in an accurate, reproducible, and repeatable manner.

To counter this, coding quality auditing system 20 uses proven statistical sampling techniques to provide clients fidelity in their coding quality and to save client money by making the coding evaluation process streamlined and more efficient. In some example approaches, coding quality auditing system 20 applies optimal sampling as described below to ensure that the work of each medical coding expert is evaluated (i.e. “sampled”) in an efficient manner without over-sampling or under-sampling. In some example approaches, coding quality auditing system 20 employs defined performance standards, providing statistical evidence that a medical coding expert is or is not performing at an acceptable customer defined performance level, thereby allowing corrective action to take place much earlier in the work performance evaluation process. In some example approaches, coding quality auditing system 20 minimizes rebuttals by defining an acceptable Margin of Error (MOE) surrounding the audit sample, reducing the need for rebuttals; only audit results outside the MOE require further inquiry or investigation. In some example approaches, coding quality auditing system 20 uses statistical attribute agreement analysis to ensure auditing standards are “calibrated” among coding auditors and are applied in an accurate, reproducible, and repeatable manner.

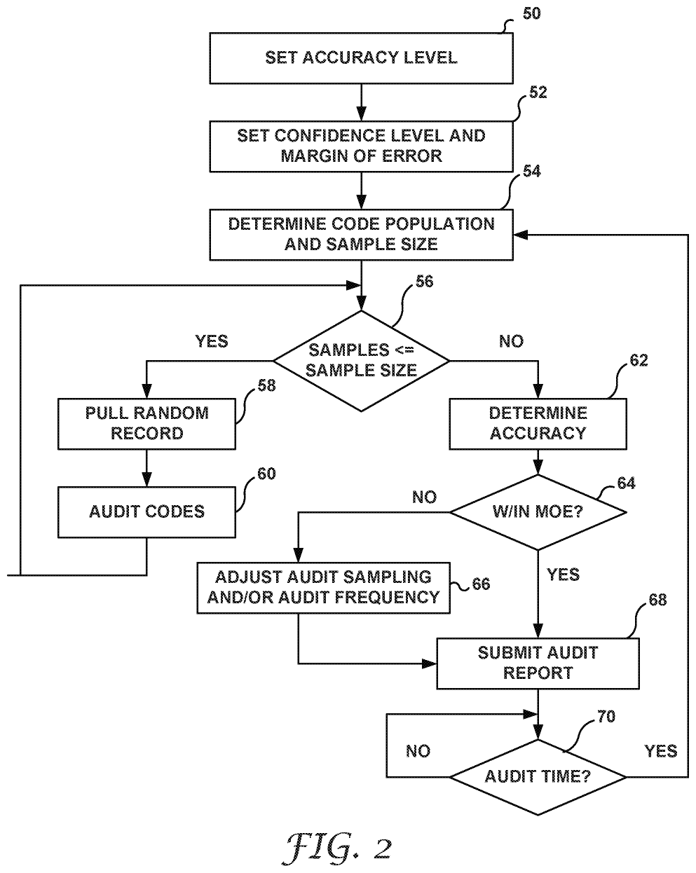

FIG. 2 illustrates an example method of auditing the performance of a medical coding expert, according to one aspect of the disclosure. In the example method of FIG. 2, a healthcare organization sets an accuracy level for each medical coding professional. (50) In some example approaches, the healthcare organization tracks the number of correct codes versus incorrect codes for each medical coding professional. In one such example approach, the accuracy level is found by dividing the number of audited codes that are correct by the total number of codes audited. The number of “records” audited is immaterial. What matters is deriving the correct number of codes to sample and then pulling the appropriate number of records to satisfy the number of codes needed in the sample size. This is a key distinction. Simply pulling a set number of records and “hoping” there are sufficient codes in the sample size is spurious logic and could result in insufficient data and low confidence in the quality scores. In some example approaches, coding quality auditing system 20 determines the number of sample codes (ICD10, E&M, and CPT) needed to measure quality to the parameters selected by the healthcare organization.

Returning to FIG. 2, the healthcare organization sets, for each medical coding professional, a confidence level and margin of error associated with the accuracy level. (52) In some example approaches, each healthcare organization sets the confidence level (CL) and the margin of error (MOE), establishing, for each medical coding professional, the statistical CL and MOE they are willing to accept. For illustrative purposes, let's assume the client healthcare organization desires a 95% CL and a 5% MOE. If so, the next step is to establish the expected competency level of the medical coding professionals being audited. In some example approaches, the healthcare organization initially assumes the competency level is at the expected standard; the required sampling is then adjusted over time depending on the proficiency of the medical coding expert until a steady state is achieved and the medical coding expert is performing at the required quality level. This approach provides the client a statistically sound basis in evaluating its coding quality and saves money as it eliminates unnecessary “over sampling.”

As noted above, the required sample size is a function of the expected quality level, the confidence level, the margin of error and the code population size. In some example approaches, the required sample size (SS) is based on:

SS=P(1−P)(Z²/E²)

where SS is the sample size, P is the expected estimate of the quality level, Z=Z_{α/2} (Z_{α/2} corresponds to the boundary of the CL) and E is the margin of error.

In some example approaches, a minimum sample size is used to capture sufficient samples in small populations:

Min(SS)=f(P,Z,E,N)

where N=Population size. In one such example approach, the minimum sample size is calculated as:

$$Min ( SS ) = \frac{N + ( E \hat{} 2 * N ) / ( Z \hat{} 2 * P * ( 1 - P ) )}{1.0 + ( E \hat{} 2 * N ) / ( Z \hat{} 2 * P * ( 1 - P ) )}$$

In some examples, system 20 may be configured to address the situation where system 20 does not have a prior estimate of the coding professional's quality level. In some example approaches, system 20 assumes that the quality level is 50%, (which is the worst case) and determines the sample size for the first audit as:

SS=0.5(1−0.5)(Z_{α/2})²/E²

or

SS=(0.25)(Z_{α/2})²/E²

The sample size for a margin of error of 5% at a 50% confidence interval would be:

SS=[(0.5)*(1−0.5)*1.96\hat{ }2]/(0.05\hat{ }2)=384.160

Rounded up the sample size is 385.

In another example approach, when system 20 first attempts to measure the accuracy of a medical coding professional, system 20 assumes that the coding professional is performing at the level desired by the healthcare organization. If we assume the organization has a coding accuracy level of 97% with a margin of error of 5% at the 95% CL:

SS=[(0.97)*(1−0.97)*1.96\hat{ }2]/(0.05\hat{ }2)=44.716

Rounded up the sample size is 45. Such an approach has the advantage of requiring a smaller sample size; system 20 may, however, need to repeat the audit process multiple times to approach a score representing the coding professional's actual quality level. In some example approaches, when system 20 first attempts to measure the accuracy of a medical coding professional, system 20 calculates a coding accuracy level for the medical coding professional at the end of each audit, repeating the audits as necessary to achieve an initial coding accuracy level for the medical coding professional.

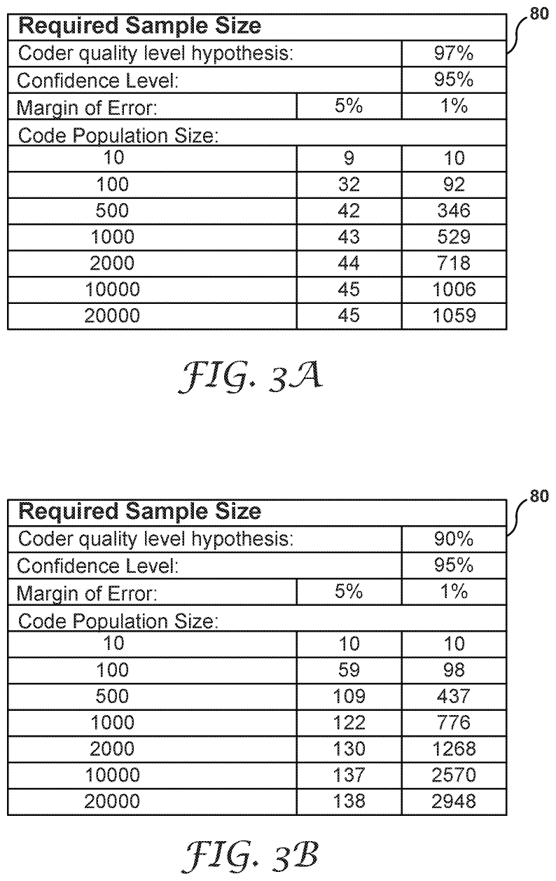

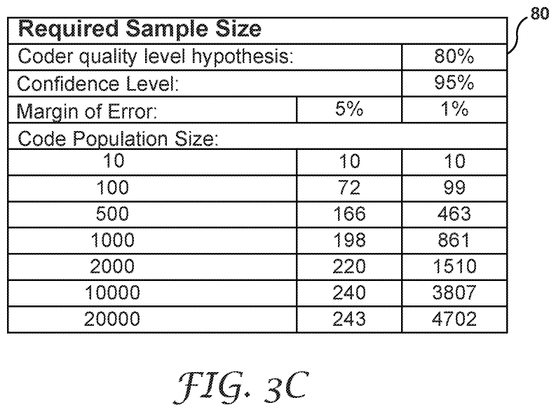

FIGS. 3A-3C illustrate a method of determining an appropriate sample size to audit a medical coding professional. In the example shown in FIG. 3A, a healthcare organization desires a coder accuracy level 80 of 97%. That is, 97 out of 100 medical code encodings are correct. Note that, in the table of FIG. 3A, the number of random samples of ICD10, E&M, and CPT codes needed to audit code populations ranging from 10 to 20,000 range from 9 to 45 samples (given our 95% CL, the 5% MOE assumptions, and the 97% coder quality hypothesis). These numbers represent examples of the number of codes to be sampled based on the estimated code population size. Note that, if the MOE assumption is changed to 1%, the number of codes required to adequately audit the code population can increase dramatically. Also note, if a coder is meeting the quality level standard it may only take a sampling of 45 codes to provide sufficient evidence of a 97% coder quality level (assuming a 95% CL and 5% MOE), even given a code population size of 20,000 or more!

In one example, Coder X is a medical coding professional for the healthcare organization. In the previous month, Coder X coded an estimated 2000 ICD10 codes, 500 E&M codes, and 100 CPT codes. In a healthcare organization that desires a confidence level of 95% and an MOE of 5%, based on the table in FIG. 3A, an auditor only must randomly select enough records to audit 44 ICD10 codes, 42 E&M codes, and 32 CPT codes to verify a 97% accuracy level.

In the example shown in FIG. 3B, a healthcare organization desires a coder accuracy level 80 of 90%. Note that, in the table of FIG. 3B, the number of random samples of ICD10, E&M, and CPT codes needed to audit code populations ranging from 10 to 20,000 range from 10 to 138 samples (given our 95% CL, the 5% MOE assumptions, and the 90% coder quality hypothesis). The reason the number of samples per code population increased is that an auditor reviewing records at an accuracy level of 90% needs more samples to verify the accuracy level. Note that, if the MOE assumption in FIG. 3B is changed to 1%, the number of codes required to adequately audit the code population can increase dramatically.

In one example, Coder X is a medical coding professional for the healthcare organization. In the previous month, Coder X coded an estimated 2000 ICD10 codes, 500 E&M codes, and 100 CPT codes. In a healthcare organization that desires a confidence level of 95% and an MOE of 5%, based on the table in FIG. 3A, an auditor now must randomly select enough records to audit 130 ICD10 codes, 109 E&M codes, and 59 CPT codes to verify a 90% accuracy level at a confidence level of 95% and an MOE of 5%.

In the example shown in FIG. 3C, a healthcare organization desires a coder accuracy level 80 of 80%. Note that, in the table of FIG. 3C, the number of random samples of ICD10, E&M, and CPT codes needed to audit code populations ranging from 10 to 20,000 range from 10 to 243 samples (given our 95% CL, the 5% MOE assumptions, and the 80% coder quality hypothesis. Note that, if the MOE assumption in FIG. 3B is changed to 1%, the number of codes required to adequately audit the code population can increase dramatically.

In one example, Coder X is a medical coding professional for the healthcare organization. In the previous month, Coder X coded an estimated 2000 ICD10 codes, 500 E&M codes, and 100 CPT codes. In a healthcare organization that desires a confidence level of 95% and an MOE of 5%, based on the table in FIG. 3A, an auditor now must randomly select enough records to audit 220 ICD10 codes, 166 E&M codes, and 72 CPT codes to verify an 80% accuracy level at a confidence level of 95% and an MOE of 5%. Once again, if the MOE assumption in FIG. 3C is changed to 10/%, the number of codes required to adequately audit the code population can increase dramatically.

Returning to FIG. 2, coding quality auditing system 20 determines a code population for each medical coding professional, and then determines a sample size for each code type based on the code population for each code type, the accuracy level, the confidence level and the margin of error associated with each of the respective medical coding professionals. (54) As noted above in FIGS. 3A-3C, the sample size for each medical coding professional may differ for each code type. In some example approaches, the sample size for each code type varies as a function of the medical coding professional's measured accuracy and of the number of codes of each code type the professional had coded since the last audit.

A check is made at 56 to determine if the random sampling of coded medical records had found enough samples of each code type to satisfy the sample size calculated for that code type for each respective medical coding professional. (56) If not, coding quality auditing system 20 pulls another randomly selected record (58) coded for that medical coding professional and determines if the codes within the record were encoded correctly (60), before returning to 56.

If, however, at 56, coding quality auditing system 20 determines the number of code samples for each code type for each medical coding professional has reached the sample size for that code type for each of the medical coding professionals, system 20 determines an accuracy level for each code type for each medical coding professional. (62) In some example approaches, the accuracy level is found by dividing the number of correct codes by the total number of codes of that type samples by system 20.

A check is made at 64 to determine if the medical coding professional has an accuracy level within the MOE defined for the medical coding professional. If so, system 20 submits an audit report to the healthcare organization for the medical coding professional (68) and waits for the next audit time (70). In some example approaches, reports and findings are submitted in the frequency, level of detail, and format desired by the healthcare organization. In some example approaches, audit records are recorded in a secure database at the security level determined by the healthcare organizations needs and requirements.

By defining an MOE for each medical coding professional, we avoid quibbling over accuracy scores that are close to the desired standard but that don't quite make the desired accuracy level. Such an approach reduces the number of rebuttal procedures. On detecting the next audit time for one or more medical coding professionals, system 20 returns to 54 to begin the next audit.

In some example approaches, each medical coding professional may have a different audit frequency, with those medical coding professionals that demonstrate higher quality work tested more infrequently than those that demonstrate lower quality performances during audits. When the next audit time occurs, system 20 returns to 54, determines the code population and determines the sampling size for the code population based on the measured accuracy level. (54) In some example approaches, the amount of subsequent audit sampling is adjusted based on the audit results. If a coder is performing at a high level and is meeting the customer defined quality standard on a consistent basis, the sample size can be reduced (thereby reducing cost). However, if the coder is not meeting the standard, a decision needs to be made to either increase the sample size and reevaluate the coder's borderline performance (i.e. monitor/trend) or take immediate action (i.e. retraining, re-education, etc.). Sometimes, the coder's results are so poor that it is extremely unlikely their performance is attributable to simple randomness and the healthcare organization must evaluate the person's fitness for the medical coding position.

In some example approaches, audit results are shared with the coders for feedback, education, and training purposes. A key attribute of the methodology described herein is the need to counsel coders who fall below the customer established quality level. This need is dramatically reduced as the focus is only on coders who have a quality score outside the statistical sampling margin of error. Example: if the quality level is 97% and the MOE is 5% (i.e. 97%+/−5%), then only coders who score below 92% warrant further inquiry or investigation.

Returning to FIG. 2, if the medical coding professional has an accuracy level outside the MOE defined for the medical coding professional, system 20 adjusts the sampling size for the medical coding professional to the appropriate sampling size for the measured accuracy level and may increase the frequency in which the medical coding professional is audited. (66) System 20 then submits an audit report to the healthcare organization for the medical coding professional (68) and waits for the next audit time (70). On detecting the next audit time for one or more medical coding professionals, system 20 returns to 54 to begin the next audit.

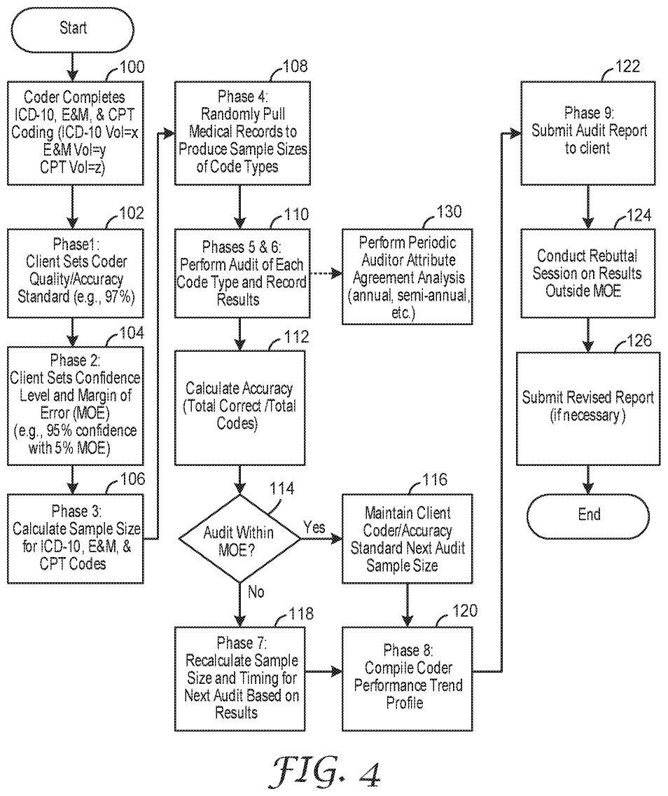

FIG. 4 is a flowchart illustrating an example method of auditing the performance of a medical coding expert, according to one aspect of the disclosure. At 100, the coding professional completes work on coding of medical records and the audit begins. In the example shown, the coding professional coded records with a code types ICD-10, E&M and CPT. The records coded included x ICD-10 codings, y E&M codings and z CPT codings. As can be seen in FIG. 4, the healthcare organization sets the coder quality/accuracy standard. (102) In other example approaches, the coder quality/accuracy standard may be set by an organization outside the healthcare organization, or may reflect best practices. The healthcare organization also sets a confidence level (representing the uncertainty of the sampling method) and the margin of error. (104)

Auditing system 20 calculates an appropriate sample size for the random sampling, for each coding type, of the medical records. (106) In some example approaches, the sample size is based on the coder quality level estimated for the coding professional being audited, the number of codings of each coding type the coding professional performed since the last audit, the selected confidence level and the selected MOE. In the example shown in FIG. 4, the result is a sample size for each of the three coding types.

In some example approaches, auditing system 20 randomly pulls enough medical records to produces a sample size number of codings of the ICD-10 code type, the E&M code type and the CPT code type. (108) An auditor reviews the selected medical records, determines if the coding was correct, and records the audit results. (110) System 20 then calculates coder accuracy for each of the three code types (112) and determines if coder accuracy is within the defined margin of error. (114) In some example approaches, if coder accuracy is within the margin of error, system 20 maintains the same coder accuracy level. (116) If a coder consistently performs at a high level, meeting the customer defined quality standard on a consistent basis, the sample size can be reduced (thereby, reducing audit cost).

In some example approaches, auditing system 20 randomly pulls enough medical records to produces a sample size number of codings of the ICD-10 code type, the E&M code type and the CPT code type. (108) An auditor reviews the selected medical records, determines if the coding was correct, and records the audit results. (110) System 20 then calculates coder accuracy for each of the three code types (112) and determines if coder accuracy is within the defined margin of error. (114) In some example approaches, if coder accuracy is within the margin of error, system 20 maintains the same coder accuracy level. (116) In some example approaches, if a coder consistently performs at a high level, meeting the customer defined quality standard on a consistent basis, the auditor or the auditing system 20 may reduce the sample size of audits for that individual (thereby reducing audit cost).

If, however, the coder is not meeting the accuracy standard (i.e., it not within the MOE), the auditor or auditing system 20 may decide to either increase the sample size for future audits and reevaluate the coder's borderline performance (i.e. monitor/trend) (118) or to take immediate action (i.e. retraining, re-education, or reassignment). In some example approaches, system 20 makes this decision based on how far outside the margin of error the coder's audit results fall.

In some example approaches, system 20 maintains a coding quality trend for each coding professional. (120) The coding quality trend looks at previous audit results and produces a graphic on UI 28 indicating how the current audit results compare to past audit results.

System 20 compiles an audit report of the coding professional based on the audit results and submits the report to the healthcare organization. (122) In some example approaches, the report includes an analysis of the type of coding mistakes made and suggestions for avoiding the errors. In some example approaches, the audit report includes trend information for the individual and across the organization.

In some example approaches, system 20 accommodates rebuttal requests but limits the requests to individuals who fell outside the margin of error on their audit. In one such example approach, system 20 sets up a rebuttal session between the auditor (at user interface 28) and the coding professional (at user interface 18) in which the coding professional can attempt to rebut the marking as incorrect of one or more of the codings. (124) If the rebuttal session results in changes to the coding professional's accuracy score, system 20 updates its records and prepares and submits a new audit report to the healthcare organization. (126)

In some example approaches, system 20 performs periodic auditor attribute agreement analysis and records the results. (130) It is important to ensure coding auditors, from a statistical perspective, are evaluating coder performance in a standardized fashion and not introducing variability into the evaluation process. Medical coding, by its nature, is subjective and introduces the possibility of human error.

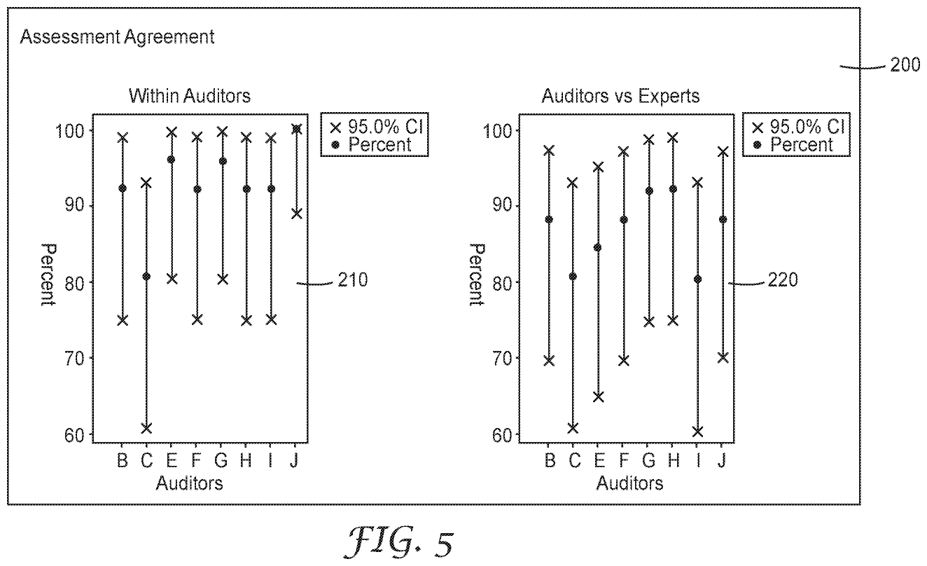

FIG. 5 illustrates an attribute assessment agreement, according to one aspect of the disclosure. In some example approaches, as detailed in FIG. 5, system 20 performs measurement system analysis (MSA) of its coders on a periodic basis by utilizing an “Attribute Agreement Analysis” tool to ensure auditors are “calibrated” and consistently scoring coders the same way. In one such example approach, as is shown in FIG. 5, attribute agreement 200 includes chart 210 that shows how consistent the auditors are within themselves in determining coding errors and a chart 220 that shows how consistent the auditors are in correctly determining coding errors.

For instance, auditors B, C, E, F, G, H, I, and J may indicate different levels of performance of ICD-10 codings; chart 210 shows how consistent the group of auditors are in making the same repeated determination of ICD-10 codings. In the example shown in FIG. 5, chart 220 shows how consistent the auditors are in getting the correct answer in each class of ICD-10 codings. Chart 220 will always be equal to or less than chart 210. This systematic use of an internal MSA detects if there is a prevalence for human error by assessing whether an auditor is performing as expected or if they are an outlier requiring additional training to bring them back to expected norms. In some example approaches, system 200 maintains a score for each auditor with a numerical result reflecting the auditor's results versus the results shown in charts 210 and 220.

### Example 1

A method of auditing medical records to determine the coding accuracy of medical coding professionals comprises assigning an expected coding accuracy level to each of a plurality of medical coding professionals; establishing a confidence level and a margin of error around each expected coding accuracy level; determining a code population for each medical coding professional and for each of one or more code types, each respective code population including codes that are subject to audit, that are of the same code type and that were assigned by the respective medical coding professional, the code population having a size that represents the number of codes therein; determining a sample size SS(i) for each medical coding professional i for each code population, the sample size based on the expected coding accuracy level, the confidence level and the margin of error associated with the respective medical coding professional; randomly retrieving medical records to obtain, for audit, approximately SS(i) samples of codes assigned by each respective medical coding professional i; determining if the codes obtained from the retrieved medical records were assigned correctly; and calculating an accuracy for each medical coding professional based on the number of correct code assignments by each respective medical coding professional.

### Example 2

The method of example 1, wherein one or more of the expected coding accuracy level, the confidence level and the margin of error associated with one of the medical coding professionals changes as a function of code type; and wherein determining a sample size SS(i) for each medical coding professional i for each code population includes determining a different SS(ij) for each medical coding professional i and for each code type j, the sample size based on the expected coding accuracy level for the respective code type and the respective medical coding professional, the confidence level for the respective code type and the respective medical coding professional and the margin of error for the respective code type and the respective medical coding professional.

### Example 3

The method of any of examples 1 and 2, wherein the accuracy for each medical coding professional is based on the number of correct code assignments by each respective medical coding professional of the respective code type divided by the number of codes of the respective code type obtained for the audit for the respective medical coding professional.

### Example 4

The method of any of examples 1-3, wherein calculating an accuracy includes calculating a new expected coding accuracy level for one of the medical coding professionals based on the accuracy calculated for the respective medical coding professional.

### Example 5

The method of any of examples 1-4, wherein calculating a new expected coding accuracy level includes calculating a new margin of error for one of the medical coding professionals based on the accuracy calculated for the respective medical coding professional.

### Example 6

The method of any of examples 1-5, wherein calculating an accuracy includes calculating a new margin of error for one or more of the medical coding professionals based on the accuracy calculated for the respective medical coding professional.

### Example 7

The method of any of examples 1-6, wherein calculating a new margin of error includes increasing the margin of error for medical coding professionals that are within a selected margin of error of a selected coding accuracy level.

### Example 8

The method of any of examples 1-7, wherein calculating a new margin of error includes increasing the margin of error for medical coding professionals that consistently fall within the margin of error established for the respective medical coding professional.

### Example 9

The method of any of examples 1-8, wherein calculating a new margin of error includes decreasing the margin of error for medical coding professionals that consistently fall outside the margin of error established for the respective medical coding professional.

### Example 10

The method of any of examples 1-9, wherein calculating an accuracy includes calculating a new expected coding accuracy level for one of the medical coding professionals based on a weighted function of the accuracy calculated for the respective medical coding professional and coding accuracy levels previously assigned to the respective medical coding professional.

### Example 11

The method of any of examples 1-10, wherein each code population includes codes of the respective code type assigned by the respective medical coding professional since a previous audit.

### Example 12

The method of any of examples 1-11, wherein the method further comprises determining when to audit based on an audit frequency assigned to each medical coding professional.

### Example 13

The method of any of examples 1-12, wherein each code population includes codes of the respective code type assigned by the respective medical coding professional since a previous audit.

### Example 14

The method of any of examples 1-13, wherein calculating an accuracy includes calculating a new audit frequency for one of the medical coding professionals based on the calculated accuracy for the respective medical coding professional.

### Example 15

The method of any of examples 1-14, wherein calculating the new audit frequency includes increasing the audit frequency for medical coding professionals that fall outside the margin of error established for the respective medical coding professional.

### Example 16

The method of any of examples 1-15, wherein determining the sample size for each code population includes calculating SS as a function of the expected coding accuracy level P, where

SS=P(1−P)(Z_{α/2})²/E²

where Z_{α/2} corresponds to the boundary of the confidence level and where E is the margin of error.

### Example 17

The method of any of examples 1-16, wherein the sample size for each code population is based on the size of the code population for the respective medical coding professional for each of the one or more code types, the expected coding accuracy level, the confidence level and the margin of error associated with the respective medical coding professional.

### Example 18

The method of any of examples 1-17, wherein determining the sample size for each code population includes calculating SS as:

$$SS = \frac{N + ( E \hat{} 2 * N ) / ( Z \hat{} 2 * P * ( 1 - P ) )}{1.0 + ( E \hat{} 2 * N ) / ( Z \hat{} 2 * P * ( 1 - P ) )}$$

where N is the code population size for the code population being audited, where P is the expected coding accuracy level, where Z=Z_{α/2}, where Z_{α/2} corresponds to the boundary of the confidence level for the respective medical coding professional, and where E is the margin of error for the respective medical coding professional.

### Example 19

The method of any of examples 1-18, wherein one or more of the expected coding accuracy level, the confidence level and the margin of error associated with one of the medical coding professionals changes as a function of code type; and wherein determining a sample size SS(i) for each medical coding professional i for each code population includes determining a different SS(i,j) for each medical coding professional i and for each code type j, the sample size based on the expected coding accuracy level for the respective code type and the respective medical coding professional, the confidence level for the respective code type and the respective medical coding professional and the margin of error for the respective code type and the respective medical coding professional.

### Example 20

The method of any of examples 1-19, wherein the code type includes all ICD-9 and all ICD-10 codes.

### Example 21

The method of any of examples 1-20, wherein the code type includes all ICD, E&M and CPT codes.

### Example 22

The method of any of examples 1-21, wherein the codes include a plurality of code types.

### Example 23

The method of any of examples 1-22, wherein assigning an expected coding accuracy level to each of a plurality of medical coding professionals includes assigning a first coding accuracy level to those medical coding professionals without an assigned coding accuracy level, the first coding accuracy level based on a minimal acceptable coding accuracy level.

### Example 24

The method of any of examples 1-23, wherein the plurality of medical coding professionals are part of an organization, wherein assigning an expected coding accuracy level to each of a plurality of medical coding professionals includes assigning a desired coding accuracy level to those medical coding professionals without an assigned coding accuracy level, the desired coding accuracy level based a coding accuracy level desired by the organization.

### Example 25

The method of any of examples 1-24, wherein determining if the codes obtained from the retrieved medical records were assigned correctly includes assigning a plurality of auditors to review the codes obtained for audit that were assigned by each respective medical coding professional; determining, by each auditor, if the codes obtained from the retrieved medical records were assigned correctly; and reviewing discrepancies in audit results between the auditors.

### Example 26

The method of any of examples 1-25, wherein determining if the codes obtained from the retrieved medical records were assigned correctly includes storing, as audit results, a record of codes determined to be assigned correctly and a record of codes determined to be assigned incorrectly; and randomly auditing the audit results for accuracy.

### Example 27

The method of any of examples 1-26, wherein determining if the codes obtained from the retrieved medical records were assigned correctly includes storing, as audit results, a record of codes determined to be assigned correctly and a record of codes determined to be assigned incorrectly; and randomly auditing the audit results for consistency.

### Example 28

A computer-readable medium comprising instructions for causing a programmable processor to assign an expected coding accuracy level to each of a plurality of medical coding professionals; establish a confidence level and a margin of error around each expected coding accuracy level; determine a code population for each medical coding professional and for each of one or more code types, each respective code population including codes that are subject to audit, that are of the same code type and that were assigned by the respective medical coding professional, the code population having a size that represents the number of codes therein; determine a sample size SS(i) for each medical coding professional i for each code population, the sample size based on the expected coding accuracy level, the confidence level and the margin of error associated with the respective medical coding professional; randomly retrieve medical records to obtain, for audit, approximately SS(i) samples of codes assigned by each respective medical coding professional i; determine if the codes obtained from the retrieved medical records were assigned correctly; and calculate an accuracy for each medical coding professional based on the number of correct code assignments by each respective medical coding professional.

### Example 29

A computer-readable medium comprising instructions for causing a programmable processor to perform any of the methods of examples 1-27.

### Example 30

An auditing system, comprising a memory, a network interface, and at least one processor connected to the memory and the network interface, wherein the memory includes instructions that, when executed by the at least one processor, cause the processor to audit medical records to determine the coding accuracy of medical coding professionals, wherein the auditing includes assigning an expected coding accuracy level to each of a plurality of medical coding professionals; establishing a confidence level and a margin of error around each expected coding accuracy level; determining a code population for each medical coding professional and for each of one or more code types, each respective code population including codes that are subject to audit, that are of the same code type and that were assigned by the respective medical coding professional, the code population having a size that represents the number of codes therein; determining a sample size SS(i) for each medical coding professional i for each code population, the sample size based on the expected coding accuracy level, the confidence level and the margin of error associated with the respective medical coding professional; randomly retrieving medical records to obtain, for audit, approximately SS(i) samples of codes assigned by each respective medical coding professional i; determining if the codes obtained from the retrieved medical records were assigned correctly; and calculating an accuracy for each medical coding professional based on the number of correct code assignments by each respective medical coding professional.

### Example 31

The auditing system of example 30, wherein the memory includes instructions that, when executed by the at least one processor, cause the processor to establish, via the network interface, a connection across a network to a medical coding system having a document database, wherein randomly retrieving medical records includes reading the medical records from the document database.

### Example 32

The auditing system of any of examples 30 and 31, wherein the memory includes instructions that, when executed by the at least one processor, cause the processor to establish, via the network interface, a connection across a network to a medical document system having a medical coding system and a document database, wherein randomly retrieving medical records includes reading the medical records from the document database.

### Example 33

The auditing system of any of examples 30-32, wherein portions of the document database are stored in cloud-based storage.

### Example 34

A computer system having at least one processor and memory comprising functional modules programmed to carry out any of the methods of examples 1-27.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media, which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. It should be understood, however, that computer-readable storage media and data storage media do not include connections, carrier waves, signals, or other transient media, but are instead directed to non-transient, tangible storage media. Disk and disc, as used, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor”, as used may refer to any of the foregoing structure or any other structure suitable for implementation of the techniques described. In addition, in some aspects of the approach, the functionality described may be provided within dedicated hardware and/or software modules. Also, the techniques could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

It is to be recognized that depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently, e.g., through multi-threaded processing, interrupt processing, or multiple processors, rather than sequentially.

In some examples, a computer-readable storage medium includes a non-transitory medium. The term “non-transitory” indicates, in some examples, that the storage medium is not embodied in a carrier wave or a propagated signal. In certain examples, a non-transitory storage medium stores data that can, over time, change (e.g., in RAM or cache).

Various embodiments of the invention have been described. These and other embodiments are within the scope of the following claims.

1. A method of auditing medical records to determine the coding accuracy of medical coding professionals, the method comprising:
assigning an expected coding accuracy level to each of a plurality of medical coding professionals;
establishing a confidence level and a margin of error around each expected coding accuracy level;
determining a code population for each medical coding professional and for each of one or more code types, each respective code population including codes that are subject to audit, that are of the same code type and that were assigned by the respective medical coding professional, the code population having a size that represents the number of codes therein;
determining a sample size SS(i) for each medical coding professional i for each code population, the sample size based on the expected coding accuracy level, the confidence level and the margin of error associated with the respective medical coding professional;
randomly retrieving medical records to obtain, for audit, approximately SS(i) samples of codes assigned by each respective medical coding professional i;
determining if the codes obtained from the retrieved medical records were assigned correctly; and
calculating an accuracy for each medical coding professional based on the number of correct code assignments by each respective medical coding professional.

2. The method of claim 1, wherein calculating an accuracy includes calculating a new expected coding accuracy level for one of the medical coding professionals based on the accuracy calculated for the respective medical coding professional.

3. The method of claim 1, wherein calculating an accuracy includes calculating a new margin of error for one or more of the medical coding professionals based on the accuracy calculated for the respective medical coding professional.

4. The method of claim 1, wherein calculating an accuracy includes calculating a new expected coding accuracy level for one of the medical coding professionals based on a weighted function of the accuracy calculated for the respective medical coding professional and coding accuracy levels previously assigned to the respective medical coding professional.

5. The method of claim 1, wherein the method further comprises determining when to audit based on an audit frequency assigned to each medical coding professional.

6. The method of claim 5, wherein calculating an accuracy includes calculating a new audit frequency for one of the medical coding professionals based on the calculated accuracy for the respective medical coding professional.

7. The method of claim 6, wherein calculating the new audit frequency includes increasing the audit frequency for medical coding professionals that fall outside the margin of error established for the respective medical coding professional.

8. The method of claim 1, wherein determining the sample size for each code population includes calculating SS as a function of the expected coding accuracy level P, where
SS=P(1−P)(Z_{α/2})²/E²
where Z_{α/2} corresponds to the boundary of the confidence level and where E is the margin of error.

9. The method of claim 1, wherein the sample size for each code population is based on the size of the code population for the respective medical coding professional for each of the one or more code types, the expected coding accuracy level, the confidence level and the margin of error associated with the respective medical coding professional.

10. The method of claim 1, wherein determining the sample size for each code population includes calculating SS as:
$$SS = \frac{N + ( E \hat{} 2 * N ) / ( Z \hat{} 2 * P * ( 1 - P ) )}{1.0 + ( E \hat{} 2 * N ) / ( Z \hat{} 2 * P * ( 1 - P ) )}$$
where N is the code population size for the code population being audited, where P is the expected coding accuracy level, where Z=Z_{α/2}, where Z_{α/2} corresponds to the boundary of the confidence level for the respective medical coding professional, and where E is the margin of error for the respective medical coding professional.

11. The method of claim 1, wherein the plurality of medical coding professionals are part of an organization, wherein assigning an expected coding accuracy level to each of a plurality of medical coding professionals includes assigning a desired coding accuracy level to those medical coding professionals without an assigned coding accuracy level, the desired coding accuracy level based a coding accuracy level desired by the organization.

12. The method of claim 1, wherein determining if the codes obtained from the retrieved medical records were assigned correctly includes:
storing, as audit results, a record of codes determined to be assigned correctly and a record of codes determined to be assigned incorrectly; and
randomly auditing the audit results for accuracy.

13. The method of claim 1, wherein determining if the codes obtained from the retrieved medical records were assigned correctly includes:
storing, as audit results, a record of codes determined to be assigned correctly and a record of codes determined to be assigned incorrectly; and
randomly auditing the audit results for consistency.

14. An auditing system, comprising:
a memory;
a network interface; and
at least one processor connected to the memory and the network interface, wherein the memory includes instructions that, when executed by the at least one processor, cause the processor to audit medical records to determine the coding accuracy of medical coding professionals, wherein the auditing includes:
assigning an expected coding accuracy level to each of a plurality of medical coding professionals;
establishing a confidence level and a margin of error around each expected coding accuracy level;
determining a code population for each medical coding professional and for each of one or more code types, each respective code population including codes that are subject to audit, that are of the same code type and that were assigned by the respective medical coding professional, the code population having a size that represents the number of codes therein;
determining a sample size SS(i) for each medical coding professional i for each code population, the sample size based on the expected coding accuracy level, the confidence level and the margin of error associated with the respective medical coding professional;
randomly retrieving medical records to obtain, for audit, approximately SS(i) samples of codes assigned by each respective medical coding professional i;
determining if the codes obtained from the retrieved medical records were assigned correctly; and
calculating an accuracy for each medical coding professional based on the number of correct code assignments by each respective medical coding professional.

15. The auditing system of claim 14, wherein the memory includes instructions that, when executed by the at least one processor, cause the processor to establish, via the network interface, a connection across a network to a medical document system having a medical coding system and a document database, wherein randomly retrieving medical records includes reading the medical records from the document database.
